(19) United States
(12) Patent Application Publication
(10) Pub. No.: US 2026/0265196 A1
(43) Pub. Date: Sep. 10, 2026

(54) MECHANOCHEMICAL PROCESS FOR SYNTHESISING A CYANINE COMPOUND

(71) Applicant: University of Sussex, Sussex (GB)

(72) Inventors: Barnaby GREENLAND, Sussex (GB); Mary WONG, Sussex (GB)

(21) Appl. No.: 19/165,698

(22) PCT Filed: Mar. 15, 2024
(86) PCT No.: PCT/EP2024/056998

§ 371 Date: Sep. 16, 2025

(30) Foreign Application Priority Data
Mar. 16, 2023 (GB) 2303864.9

(51) Int. Cl. C07D 209/62

(52) U.S. Cl. CPC C07D 209/62

(57) ABSTRACT

The present invention provides a mechanochemical process for synthesising a cyanine compound of Formula (I), such as indocyanine green. The process is carried out in the absence of solvent.

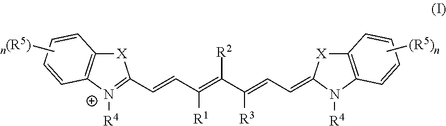

### FIELD OF THE INVENTION

The present invention provides a mechanochemical process for synthesising a cyanine compound such as indocyanine green.

### BACKGROUND

Cyanine dyes such as indocyanine green (ICG) are useful in medical diagnostics. For example, ICG is a fluorescent dye which is used in medicine as an indicator substance, for example to measure cardiac output, liver function, or in ophthalmic angiography. It is administered intravenously and is generally eliminated from the body with a half life of about 3 to 4 minutes.

Until now, ICG has been made using variants of multi-step, solvent-heavy synthesis, for example as described in US 2019/0337896 A1. It is, however, desired to provide further and improved methods of synthesis.

### SUMMARY OF THE INVENTION

The present invention provides a new synthetic process for the production of cyanine dyes such as indocyanine green (ICG). The process of the present invention is a mechanochemical process. That is, the chemical reaction underlying the process is induced by mechanical energy. Consequently, the process of the present invention is generally performed in the solid state, i.e. using solid starting materials. Furthermore, the process is generally carried out in the absence of any solvent.

Thus, in one aspect the invention provides a mechanochemical process for synthesising a cyanine compound of Formula (I) or a salt thereof:

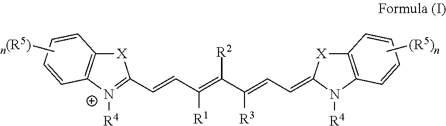

wherein:
   R¹ and R³ are each independently selected from the group consisting of H, OH, C₁₋₃ methyl, halo, phenyl; or R¹ and R³ are joined together to form —CH₂—CH₂— or —CH₂—CR₂—CH₂—, where R is H or CH₃;
   R² is H, halo, —CN, —OH, —COOH, —COOCH₃, C₁₋₆ alkyl, C₅₋₆ aryl, or N(R⁶)₂, where R⁶ is H, C₁₋₆ methyl, or phenyl;
   each R⁴ is independently selected from the group consisting of —C₁₋₅ alkyl, which may be optionally substituted by —COOH or —SO₃H;
   each R⁵ is independently selected from the group consisting of H, halo, —OH, —COOH, —SO₃H, or C₁₋₆ methyl, or two R⁵ groups on adjacent carbons together form a phenyl ring;
   each n is independently 0, 1 or 2; and
   each X is independently selected from the group consisting of S, CH₂ or C(CH₃)₂; the process comprising mechanochemically reacting a compound of Formula (II) or a salt thereof with one or more compounds of Formula (III) in the presence of a catalyst of Formula (IV) and a base,
      wherein the compounds of Formulae (II) and (III) and the catalyst of Formula (IV) have the following structures:

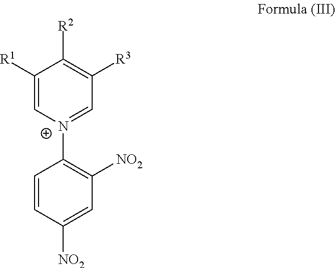

wherein R¹, R² and R³ are as defined for Formula (I);

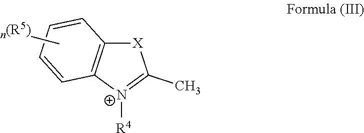

wherein n, R⁴, R⁵, and X are as defined for Formula (I);

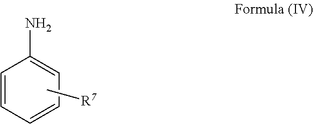

wherein R⁷ is selected from H, a halogen, C₁-C₆ alkyl, C₁-C₆ alkoxy and phenoxy.

### FIGURES

FIG. 1 shows the chemical structure of indocyanine green (ICG).

### DETAILED DESCRIPTION

As discussed above, the present invention provides a new and improved method for the synthesis of cyanine dyes such as indocyanine green (ICG). Since the process of the present invention is a mechanochemical process, it may be done in the absence of solvent, and as a continuous process. This is advantageous over known synthetic processes which involve a standard batch-type process, where starting materials are dissolved in a solvent which is contained within glassware, the reaction runs for many hours and the product is purified at the end, generally resulting in product formation once a day. The solvent is then discarded, causing waste. In addition, the equipment must be cleaned and the reaction restarted.

In contrast, the synthetic process described herein may be done as a continuous process, for example in either a double- or single-screw extruder. When using an extruder, the starting materials may be fed into the extruder at one end, the product forms as it passes down the extruder, and the product emerges at the other end. As long as the extruder remains ‘fed’ with starting materials then the production process can be maintained without stopping. Additionally, because the process of the invention is a mechanochemical process, it typically requires no solvent. As a result, no solvent waste is produced, making the process easier and greener. For example, the process may be potentially cheaper and more environmentally friendly than a traditional process involving the use of solvents.

The process of the present invention is therefore time, energy, space and resource efficient. It is more environmentally friendly than previous reported methods, as well as labour-saving. It also conveniently lends itself to industrial volume output.

### Formula (I)

The process of the invention is a process for synthesising a cyanine compound of Formula (I) or a salt thereof:

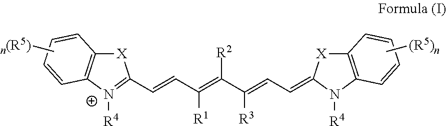

wherein:
   R¹ and R³ are each independently selected from the group consisting of H, OH, C₁₋₃ methyl, halo, phenyl; or R¹ and R³ are joined together to form —CH₂—CH₂— or —CH₂—CR₂—CH₂—, where R is H or CH₃;
   R² is H, halo, —CN, —OH, —COOH, —COOCH₃, C₁₋₆ alkyl, C₅₋₆ aryl, or N(R⁶)₂, where R⁶ is H, C₁₋₆ methyl, or phenyl;
   each R⁴ is independently selected from the group consisting of —C₁₋₅ alkyl, which may be optionally substituted by —COOH or —SO₃H;
   each R⁵ is independently selected from the group consisting of H, halo, —OH, —COOH, —SO₃H, or C₁₋₆ methyl, or two R⁵ groups on adjacent carbons together form a phenyl ring;
   each n is independently 0, 1 or 2; and
   each X is independently selected from the group consisting of S, CH₂ or C(CH₃)₂.

A counterion to the iminium group (i.e. the positively charged nitrogen) is generally present. Suitable counterions include, for example, halo ions such as Br⁻ or I⁻. Such counterions will generally be separate to the compound of Formula (I). Alternatively, the counterion may be present within structure of Formula (I). For example, as discussed further below, any of the acidic substituents (e.g. when R⁴ contains the groups —COOH or —SO₃H) may be unprotonated (e.g. —COO⁻ or —SO₃⁻).

R¹ and R³

R¹ and R³ are each independently selected from the group consisting of H, OH, C₁₋₃ methyl, halo, phenyl; or R¹ and R³ are joined together to form —CH₂—CH₂— or —CH₂—CR₂—CH₂—, where R is H or CH₃.

Preferably, R¹ and R³ are the same.

Preferably, R¹ and R³ are each independently selected from the group consisting of H, OH, C₁₋₃ methyl, Cl, Br, I, or phenyl; or R¹ and R³ are joined together to form —CH₂—CH₂—CH₂—.

Even more preferably, R¹ and R³ are both H.

### R²

R² is H, halo, —CN, —OH, —COOH, —COOCH₃, C₁₋₆ methyl, phenyl, or N(R⁶)₂, where R⁶ is H, C₁₋₆ methyl, or phenyl.

More preferably, R² is H, Br, I, —COOH, or phenyl.

Even more preferably, R² is H.

### R⁴

Each R⁴ is independently selected from the group consisting of —C₁₋₅ alkyl, which may be optionally substituted by —COOH or —SO₃H.

Preferably, each R⁴ group is the same.

Preferably, each R⁴ is independently selected from the group consisting of —C₁₋₄ alkyl, —(CH₂)_{y}—COOH or —(CH₂)_{y}—SO₃H, where y is 1, 2, 3 or 4.

More preferably, each R⁴ is independently selected from the group consisting of —CH₃, —(CH₂)₄—COOH or —(CH₂)₄—SO₃H.

Even more preferably, each R⁴ is —(CH₂)₄—SO₃H.

The process of the invention covers methods of making a cyanine compound of Formula (I) or a salt thereof. Preferred salts are sodium salts. Thus, in each case, where an R⁴ group contains an acidic group (e.g. —COOH or —SO₃H), this group may be present in protonated, unprotonated or salt (e.g. sodium salt) form.

Simply by way of example, R⁴ may be —(CH₂)₄—SO₃⁻ or —(CH₂)₄—SO₃⁻W⁺, where W is a cation such as Na⁺. Generally the R⁴ group attached to the iminium nitrogen (i.e. the positively charged nitrogen shown in Formula (I)) is unprotonated (i.e. with a negative charge), and the other R⁴ group is in salt (e.g. sodium salt) form.

### R⁵

Each R⁵ is independently selected from the group consisting of H, halo, —OH, —COOH, —SO₃H, or C₁₋₆ methyl, or two R⁵ groups on adjacent carbons together form a phenyl ring.

Preferably, each R⁵ is H or —SO₃H, or two R⁵ groups on adjacent carbons together form a phenyl ring.

Preferably, each n is the same and is 0 or 2.

As with the R⁴ groups, any acidic R⁵ groups may be present in protonated, unprotonated or salt (e.g. sodium salt) form.

More preferably, two R⁵ groups on adjacent carbons together form a phenyl ring, and are positioned such that the compound is a compound of Formula (IA)

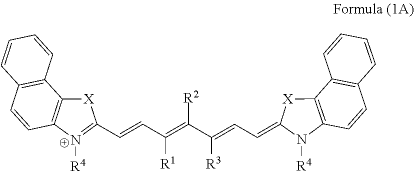

wherein R¹, R², R³, R⁴ and X are as defined herein.

### X

Each X is independently selected from the group consisting of S, CH₂ or C(CH₃)₂.

Preferably, each X is independently selected from the group consisting of S or C(CH₃)₂.

Preferably each X is the same, and more preferably each X is C(CH₃)₂.

### Definitions

As used herein, the term alkyl refers to straight and branched saturated aliphatic hydrocarbon chains. Preferred alkyl groups include methyl (Me), ethyl (Et), propyl (e.g., n-propyl and isopropyl), and butyl (e.g., n-butyl, isobutyl, t butyl).

As used herein, the term alkoxy refers to an O-alkyl group. Preferred alkoxy groups include, but are not limited to, methoxy, ethoxy, propoxy (e.g., n-propoxy and isopropoxy), and t-butoxy.

### Preferred Compounds

Preferably, the compound of Formula (I) corresponds to a compound of Formula (IA) as shown above.

More preferably, the compound of Formula (I) corresponds to a compound of Formula (IB):

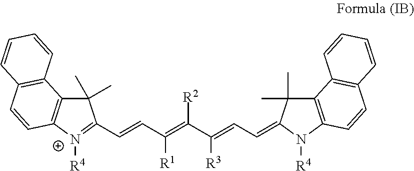

wherein R¹, R², R³ and R⁴ are as defined above.

More preferably, the compound of Formula (I) corresponds to a compound of Formula (IC):

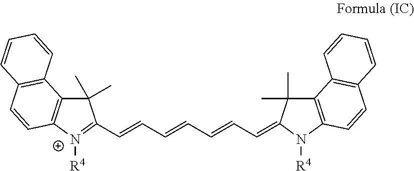

wherein each R⁴ is as defined above.

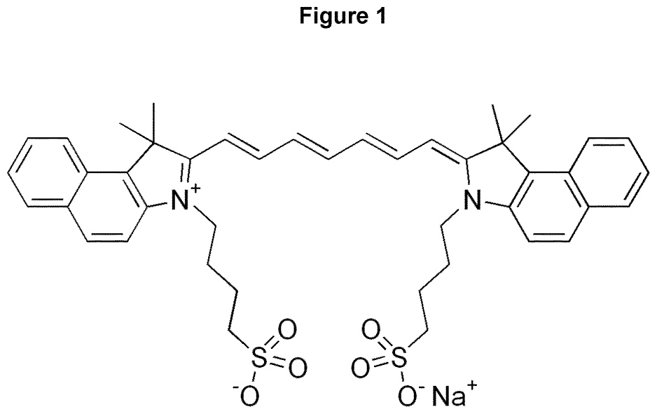

Most preferably, the compound of Formula (I) corresponds to indocyanine (ICG), having the structure shown in FIG. 1.

### Reagents

The process involves reacting mechanochemically reacting a compound of Formula (II) or a salt thereof with one or more compounds of Formula (III) in the presence of a catalyst of Formula (IV) and a base.

The structures of the compounds of Formula (II), Formula (III) and Formula (IV) are shown below. In each case the substituents (e.g. R¹, R², etc.) may have any of the definitions provided herein.

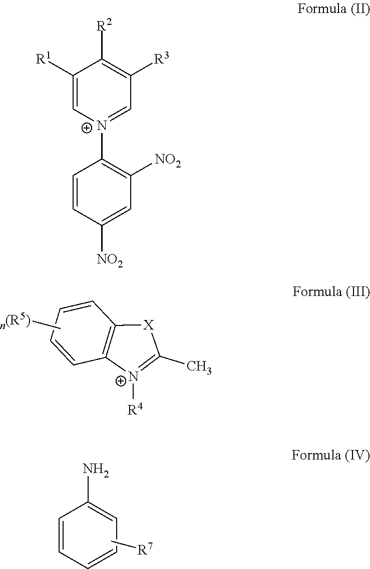

wherein R⁷ is selected from H, a halogen, C₁-C₆ alkyl, C₁-C₆ alkoxy and phenoxy.

Preferably, the R⁷ substituent is in the para position, such that the compound of Formula (IV) has the following structure:

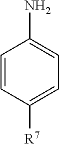

Preferably, R⁷ is selected from H, Br, C₁-C₆ alkyl, C₁-C₆ alkoxy and phenoxy.

More preferably, R⁷ is Br, such that the compound of Formula (IV) is bromoaniline.

Most preferably, R⁷ is Br and is in the para position, such that the compound of Formula (IV) is 4-bromoaniline.

In some aspects, the compound of Formula (IV) has a melting point of from about −10° C. to about 120° C., preferably from about 10° C. to about 100° C., more preferably from about 30° C. to about 80° C., and most preferably from about 50° C. to about 70° C.

As would be immediately apparent to the skilled person, the structure of the compound of Formula (I) is determined by the structure of the starting materials used in the process of the invention. To form the desired compounds the structure of the starting materials can be adjusted appropriately. Thus, to form compounds of Formula (I), IA, IB or IC having a certain substituent (e.g. R⁴) definition, starting materials (e.g. the compounds of Formula (II), Formula (III) and Formula (IV)) having the same substituent definition(s) should be used.

In the process of the invention the reagents may be present in the amount of from about 0.5 to about 2 equivalents of Formula (II); from about 1 to about 3 equivalents of Formula (III); from about 1 to about 3 equivalents of Formula (IV); and from about 2 to about 10 equivalents of base.

The reagents may alternatively be present in the amount of from about 0.8 to about 1.2 equivalents of Formula (II); from about 1.8 to about 2.2 equivalents of Formula (III); from about 1.2 to about 1.8 equivalents of Formula (IV); and from about 3 to about 9 equivalents of base (such as from about 3 to about 5 or from about 7 and about 9).

The base may act to deprotonate the compound of Formula (III). The base is preferably an acetate salt, such as sodium acetate.

### Process Conditions

The process of the present invention is a mechanochemical process, which is generally performed in the absence of any solvent. Put another way, the process of the invention may be a solid-state process.

The process of the invention may be carried out in any suitable apparatus for performing a mechanochemical process. The apparatus may be any milling, grinding or extrusion device. Suitable devices for carrying out the process of the invention include a ball mall and an extruder, which may be a single-screw or double-screw extruder. The process may also be carried out simply by heating and mixing (e.g. by stirring) the reactants together in the absence of any solvent.

Preferably the process of the invention is carried out in an extruder, more preferably a single-screw extruder. Using an extruder to carry out the process of the invention has the advantage that the process can be a continuous process, meaning that product can continually be formed.

The process of the invention is therefore a effectively a one-pot reaction process, in that all of the reagents are added to the apparatus or device in which the reaction will occur (for example a ball mill or an extruder). The reaction is then performed by operating the device. For example, by shaking the ball mill or activating the extruder.

The process of the invention may be carried out at a temperature above about 25° C., preferably above about 40° C., more preferably above about 60° C.

The process may be carried out at a temperature of from about 25° C. to about 200° C., preferably from about 40° C. to about 150° C., more preferably from about 60° C. to about 120° C.

Where the process is carried out in an extruder, the screw speed may be any suitable speed, such as from about 5 to about 500 RPM, such as from about 10 to about 250 RPM or from about 20 to about 100 RPM. The feed rate of the extruder may be, simply by way of example, from about 100 to about 1000 g/hr, such as from about 200 to about 500 g/hr.

The time for the reaction may depend on the reaction conditions and the apparatus or device in which the reaction is taking place. By way of example, the reaction may take from about 1 minute to about 4 hours, such as from about 2 minutes to about 2 hours. The skilled person would be aware of suitable methods for monitoring the progress of the reaction, including visual inspection (e.g. noting a colour change).

### EXAMPLES

### Methods and Materials

Reagents were purchased from Apollo Scientific, Carbosynth, Merck, Fisher Scientific UK Ltd, Tokyo Chemical Industry UK Ltd or Fluorochem Ltd, and used without further purification. The ball milling reactions were carried out in a Retsch MM400 vibratory ball mill (VBM) operating at 30 Hz and a Retsch PM100 planetary ball mill (PBM). Milling load is defined as the sum of the mass of the reactants per free volume in the jar. Single Screw Extrusion reactions were performed using a FilaFab PRO 350X operating at 25 rpm and variable temperatures. Pre-mixing of reagents was accomplished in a 1 L Kenwood Mini-Chopper.

Unless stated otherwise, purifications were performed via flash column chromatography on silica gel (RediSep® Rf Silica Gel Disposable Flash Columns, 40-60 micron) on Teledyne ISCO CombiFlash Lumen apparatus. Analytical thin layer chromatography (TLC) was performed on silica gel 60 F254 (Merck).

### Example 1—Synthesis of sodium;4-[(2Z)-2-[(2E,4E,6E)-7-[1,1-dimethyl-3-(4-sulfonatobutyl)benzo[e]indol-3-ium-2-yl]hepta-2,4,6-trienylidene]-1,1-dimethylbenzo[e]indol-3-yl]butane-1-sulfonate, ICG

### Example 1.1—Single Screw Extruder

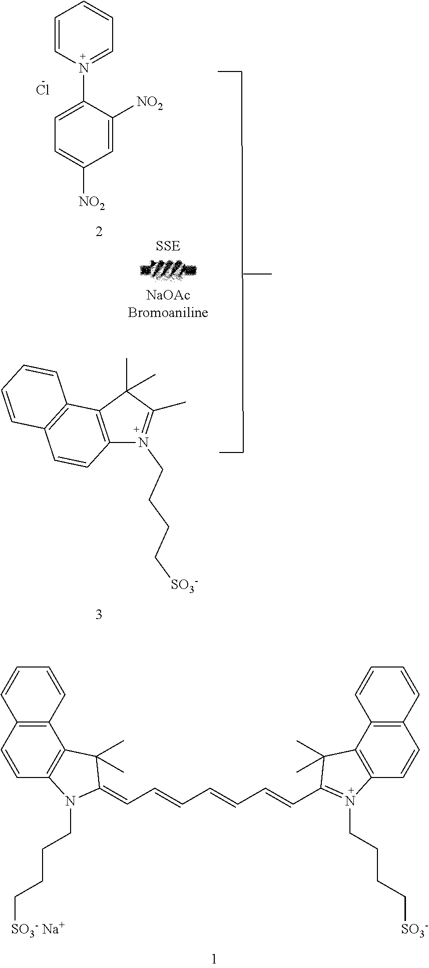

1-(2,4-Dinitrophenyl) pyridinium chloride (2) (14.3 g, 50.1 mmol), (3) 4-(1,1,2-trimethyl-1H-benzo[e]indolium-3-yl)butane-sulfonate (35.0 g, 101 mmol), 4-bromoaniline (13.0 g, 75.2 mmol) and sodium acetate (33.1 g, 404 mmol) were pre-mixed in a Kenwood mini-chopper for one minute using 5-second bursts. The SSE extruder (Filafab 350 PRO EX) was preheated to 85° C., with the screw rotation set to 25 rpm. All the contents from the mini-chopper were poured into the extruder's hopper and processed into four different stirring 50:50 mixtures of solvent. This sample was washed in IPA/acetone (1 L). The crude mixture was filtered through a glass frit and dried overnight to afford the washed crude of the title compound (11.6 g, 88.9%). A portion was purified using silica gel (4 g) column chromatography with chloroform and MeOH gradient (8:2) to give the title compound (1) as a deep green-black solid (0.50 g, 72%).

### Example 1.2—Heat and Stir

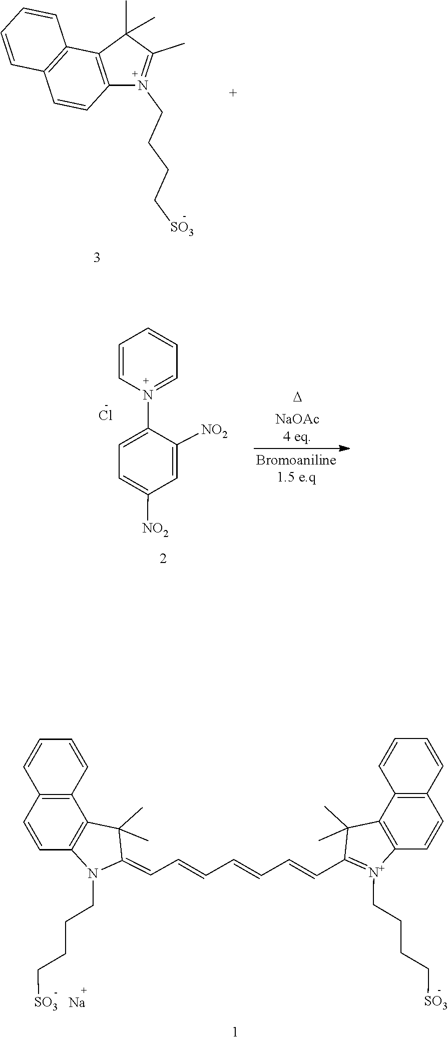

1-(2,4-Dinitrophenyl) pyridinium chloride (0.245 g, 0.9 mmol), 4-(1,1,2-trimethyl-1H-benzo[e]indolium-3-yl)butane-sulfonate (0.60 g, 1.7 mmol), 4-bromoaniline (0.232 g, 1.35 mmol) and sodium acetate (0.095 g, 3.60 mmol) were added together in a flat-bottomed conical flask (100 mL) with a 2 cm pivoted magnetic stirrer bar. It was placed on a RT hotplate set to (85° C.) and the stirrer switched on. The pale coloured reagents at RT changed to a deep green mixture within 60 seconds on the hotplate. The stirrer was immobilised by the reaction mixture at 72° C. and the mixture was manually stirred with a steel spatula. After a further four minutes on the hotplate, the flask was removed from the heat. The crude product was purified using silica gel (12 g) column chromatography with MeOH and chloroform gradient (8:2) to give the title compound (1) as a deep green solid (0.50 g, 72%).

### Example 1.3—Ball Milling

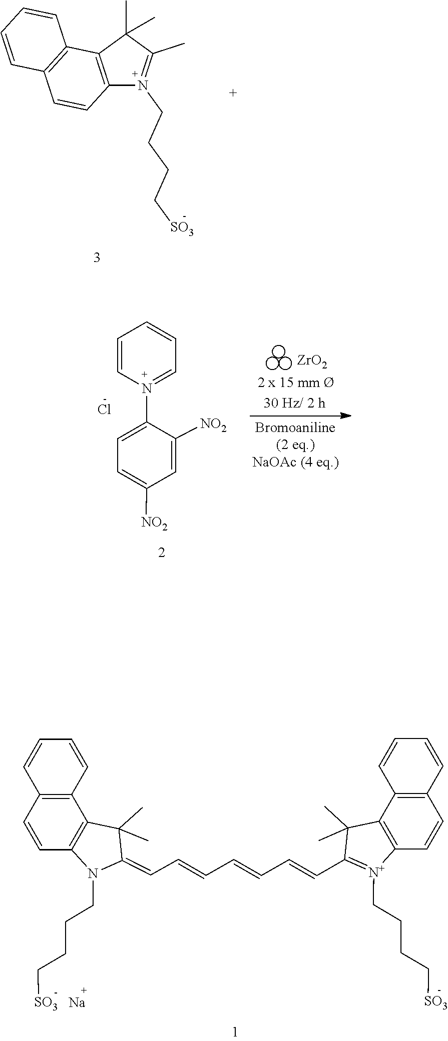

1-(2,4-Dinitrophenyl) pyridinium chloride (0.20 g, 0.70 mmol) (2), 4-(1,1,2-trimethyl-1H-benzo[e]indolium-3-yl)butane-sulfonate (0.54 g, 1.56 mmol) (3), 4-bromoaniline (0.25 g, 1.96 mmol) and sodium acetate (0.26 g, 3.12 mmol) were oscillated for 120 minutes at 30 Hz in ZrO₂ jars (25 mL) with two ZrO₂ balls (15 mm diameter). The product was dissolved in methanol (30 mL) and diethyl ether (90 mL) were added. It was kept in the freezer (−20° C.) overnight. After filtration through a chilled, glass frit, the collected solid (0.9 g) was added to acetronitrile and heated to reflux (74° C.) for an hour and cooled slowly to room temperature then filtered and washed with acetone. Dissolving in water and recrystallizing from acetone (three times) gave the title compound (3) as a green solid (0.161 g, 29%).

### Reference Example 2—Solvent-Free Synthesis of N-(2,4-Dinitrophenyl) Pyridinium Chloride

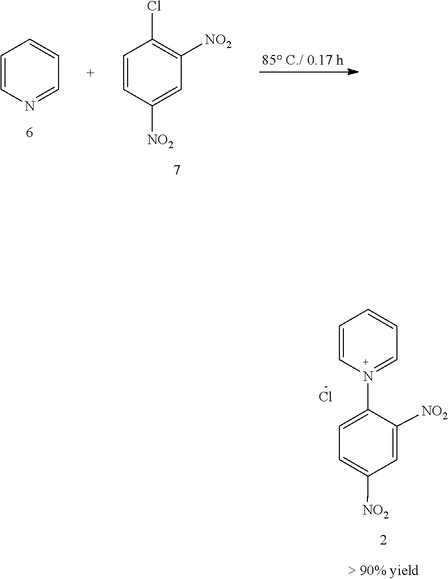

Pyridine (7.91 g, 0.100 mol) were poured into a 500 mL round-bottomed flask. 2,4-Dinitrochlorobenzene (20.3 g, 0.100 mol) was added and heated for 15 minutes with vigorous stirring. After cooling to room temperature, acetone (50 mL) was added to wash down the sides of the flask. The solid was triturated with 400 mL acetone then filtered in vacuo and washed with further acetone through a glass frit. After drying overnight under high vacuum at 65° C. to give the title compound 2 as a pale yellow solid (27.7 g, 97%).

### Reference Example 3—Synthesis of trimethyl-1-(4-sulfobutyl)-indolium inner salt (3)

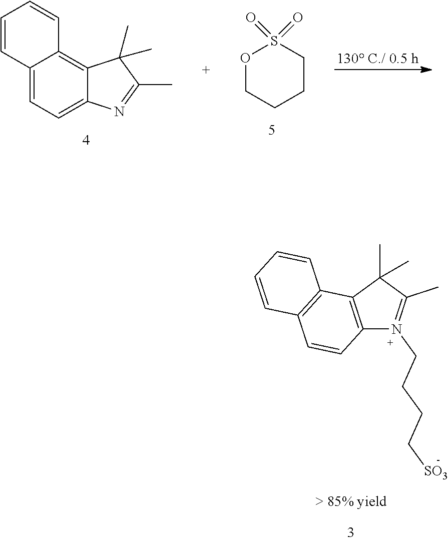

1,1,2-trimethyl-1H-benzo[e]indole (12.71 g, 60.7 mmol) were poured into a 500 mL round-bottomed flask. 1,4-Butane sultone (8.27 g, 60.7 mmol) was added and heated to reflux for 30 minutes with vigorous stirring until the stirrer bar could no longer move. While warm, DCM (400 mL) was added to creating a slurry and stirred while it cooled for 4 h to RT. After filtration the solid was washed again with DCM (400 mL), with stirring, then filtered and washed with DCM through a glass frit. It was dried overnight under high vacuum at 65° C. to give the title compound 3 as a lilac powder (18.4 g, 88%).

1. A mechanochemical process for synthesising a cyanine compound of Formula (I) or a salt thereof: 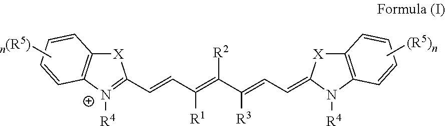
wherein:
R¹ and R³ are each independently selected from the group consisting of H, OH, C₁₋₃ methyl, halo, phenyl; or R¹ and R³ are joined together to form —CH₂—CH₂— or —CH₂—CR₂—CH₂—, where R is H or CH₃;
R² is H, halo, —CN, —OH, —COOH, —COOCH₃, C₁₋₆ alkyl, C₅₋₆ aryl, or N(R⁶)₂, where R⁶ is H, C₁₋₆ methyl, or phenyl;
each R⁴ is independently selected from the group consisting of —C₁₋₅ alkyl, which may be optionally substituted by —COOH or —SO₃H;
each R⁵ is independently selected from the group consisting of H, halo, —OH, —COOH, —SO₃H, or C₁₋₆ methyl, or two R⁵ groups on adjacent carbons together form a phenyl ring;
each n is independently 0, 1 or 2; and
each X is independently selected from the group consisting of S, CH₂ or C(CH₃)₂;
the process comprising mechanochemically reacting a compound of Formula (II) or a salt thereof with one or more compounds of Formula (III) in the presence of a catalyst of Formula (IV) and a base,
wherein the compounds of Formulae (II) and (III) and the catalyst of Formula (IV) have the following structures: 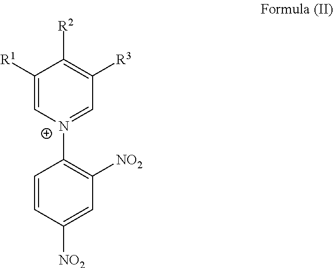
wherein R¹, R² and R³ are as defined for Formula (I); 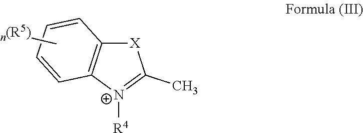
wherein n, R⁴, R⁵, and X are as defined for Formula (I); 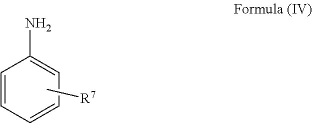
wherein R⁷ is selected from H, a halogen, C₁-C₆ alkyl, C₁-C₆ alkoxy and phenoxy; and
wherein the process is carried out in the absence of solvent.

2. The process of claim 1, wherein the process is a solid-state process.

3. (canceled)

4. The process of claim 1, wherein R¹ and R³ are the same.

5. The process of claim 1, wherein R¹ and R³ are each independently selected from the group consisting of H, OH, C₁₋₃ methyl, Cl, Br, I, or phenyl; or R¹ and R³ are joined together to form —CH₂—CH₂—CH₂—.

6. The process of claim 1, wherein R¹ and R³ are both H.

7. The process of claim 1, wherein R² is H, Br, I, —COOH, or phenyl.

8. The process of claim 1, wherein R² is H.

9. The process of claim 1, wherein each R⁴ group is the same.

10. The process of claim 1, wherein each R⁴ is independently selected from the group consisting of —C₁₋₄ alkyl, —(CH₂)_{y}—COOH or —(CH₂)_{y}—SO₃H, where y is 1, 2, 3 or 4.

11. (canceled)

12. The process of claim 1, wherein each R⁴ is —(CH₂)₄—SO₃H.

13. The process of claim 1, wherein each R⁵ is H or —SO₃H, or two R⁵ groups on adjacent carbons together form a phenyl ring.

14. The process of claim 1, wherein each n is the same and is 0 or 2.

15. The process of claim 1, wherein two R⁵ groups on adjacent carbons together form a phenyl ring.

16. The process of claim 1, wherein the compound of Formula (I) is a compound of Formula (IA)

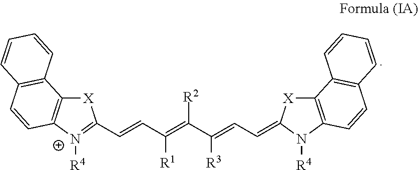

17. The process of claim 1, wherein the compound of Formula (I) is a compound of Formula (IB)

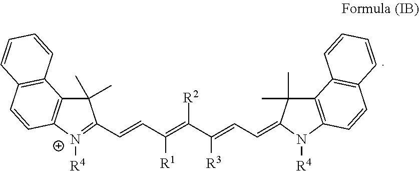

18. The process of claim 1, wherein the compound of Formula (I) is a compound of Formula (IC)

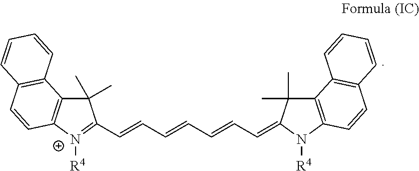

19. The process of claim 1, wherein the compound of Formula (I) is indocyanine green.

20. The process of claim 1, wherein the process is carried out in a ball mill.

21. The process of claim 1, wherein the process is carried out in an extruder.

22. (canceled)

23. (canceled)

24. The process of claim 1, wherein the process is carried out at a temperature of from about 40° C. to about 150° C.

25. (canceled)
